(19) United States
(12) Patent Application Publication
(10) Pub. No.: US 2020/0008860 A1
(43) Pub. Date: Jan. 9, 2020

(54) ENT ADVANCED ENERGY FORCEPS

(71) Applicant: COVIDIEN LP, MANSFIELD, MA (US)

(72) Inventor: GRANT T. SIMS, BOULDER, CO (US)

(21) Appl. No.: 16/433,940

(22) Filed: Jun. 6, 2019

Related U.S. Application Data
(60) Provisional application No. 62/693,537, filed on Jul. 3, 2018.

(51) Int. Cl. A61B 18/14

(52) U.S. Cl. CPC A61B 18/1442; A61B 2018/00589; A61B 18/1445

(57) ABSTRACT

A forceps includes a housing having one or more shaft members extending therefrom configured to support an end effector assembly at a distal end thereof. The end effector includes first and second pivotable jaw members each having first and second plates associated therewith. The first plates of the first and second jaw members are adapted to connect to an electrosurgical generator such that, upon activation thereof, the first plates energize tissue disposed therebetween. A knife assembly is operably associated with the housing and is configured to advance a knife through a knife channel defined through the first and second plates.

### CROSS REFERENCE TO RELATED APPLICATION

The present application claims the benefit of and priority to U.S. Provisional Application Ser. No. 62/693,537, filed on Jul. 3, 2018 the entire contents of which are incorporated herein by reference.

### BACKGROUND

### Technical Field

The present disclosure relates to surgical instruments and, more particularly, to an open or laparoscopic surgical forceps for use with various types of tissue treatments such as tonsillectomies.

### Description of Related Art

The tonsils and adenoids are part of the lymphatic system and are generally located in the back of the throat. These parts of the lymphatic system are generally used for sampling bacteria and viruses entering the body and activating the immune system when warranted to produce antibodies to fight oncoming infections. More particularly, the tonsils and adenoids break down the bacteria or virus and send pieces of the bacteria or virus to the immune system to produce antibodies for fighting off infections. As a result of inflammation of the tonsils or adenoids, a surgeon may need to perform a tonsillectomy and/or adenoidectomy.

Typically during these procedure, a variety of instruments are exchanged or “swapped out” by the surgeon several times during the course of a procedure which can be time consuming and, generally, more expensive. For example, one instrument may be utilized for fine dissection, another for nerve or tissue monitoring and a third for treating tissue (coagulating or sealing tissue).

Other procedures may require similar instrument exchanges, e.g., plastic or reconstructive surgery, thyroidectomy, parathyroidectomy, parotidectomy (removal of the salivary gland), neck dissection, lymph node dissection, facial dissection, etc.

### SUMMARY

As used herein, the term “distal” refers to the portion that is being described which is further from a user, while the term “proximal” refers to the portion that is being described which is closer to a user.

In accordance with one aspect of the present disclosure, a forceps includes a housing have one or more shaft members extending therefrom configured to support an end effector assembly at a distal end thereof. The end effector includes first and second opposing jaw members each including a first plate associated with a first portion of the jaw member and a second plate associated with a second portion of the jaw member. One or both of the first and second jaw members is pivotable relative to the other about a pivot such that the jaw members are selectively movable between an open position wherein the jaw members are spaced relative to one another and a closed position for grasping tissue therebetween. The first and second plates of the first and second jaw members are disposed in opposition relative to one another and the first plates of the first and second jaw members are adapted to connect to an electrosurgical generator such that, upon activation thereof, the first plates energize tissue disposed therebetween. A knife assembly is operably associated with the housing and is configured to selectively advance a knife through a knife channel defined in one or both of the jaw members. The knife is selectively advanceable through both the first and second plates of one or both of the jaw members.

In aspects according to the present disclosure, the second plate is non-conductive. In other aspects, an insulator is included that is disposed between the first and second plates of one or both jaw members. In still other aspects according to the present disclosure, the second plate is adapted to connect to a tissue monitoring system.

In yet other aspects according to the present disclosure, a first switch is included that is adapted to connect to the electrosurgical generator and is configured to energize the first opposing plates on the first and second jaw members to seal tissue upon activation thereof, and a second switch is included that is adapted to connect to a tissue monitoring system and is configured to obtain feedback from the tissue disposed between the second opposing plates on the first and second jaw members.

In aspects, the tissue monitoring system provides feedback relating to nerves, critical tissue structures, and/or tissue type.

In still other aspects according to the present disclosure, a first switch is adapted to connect to a first mode of the electrosurgical generator and is configured to energize the first opposing plates on the first and second jaw members to seal tissue upon activation thereof, and a second switch is adapted to connect to a second mode of the electrosurgical generator and is configured to coagulate tissue disposed between the second opposing plates on the first and second jaw members upon activation thereof.

In accordance with another aspect of the present disclosure, a forceps includes a housing having one or more shaft members extending therefrom configured to support an end effector assembly at a distal end thereof. The end effector includes first and second opposing jaw members each having a first electrically conductive plate associated with a first portion of the jaw member and a second electrically conductive plate associated with a second portion of the jaw member. One or both of the first and second jaw members is pivotable relative to the other about a pivot such that the jaw members are selectively movable between an open position wherein the jaw members are spaced relative to one another and a closed position for grasping tissue therebetween. The first and second electrically conductive plates of the first and second jaw members are disposed in opposition relative to one another. A first switch is included that is adapted to connect to a first mode of an electrosurgical generator and is configured to energize the first opposing electrically conductive plates on the first and second jaw members to seal tissue upon activation thereof. A second switch is included that is adapted to connect to a tissue monitoring system and is configured to obtain feedback from the tissue disposed between the second opposing electrically conductive plates on the first and second jaw members. A third switch is included that is adapted to connect to a second mode of the electrosurgical generator and is configured to energize the second opposing electrically conductive plates on the first and second jaw members to coagulate tissue upon activation thereof.

In other aspects according to the present disclosure, a knife assembly is included that is operably associated with the housing and is configured to selectively advance a knife through a knife channel defined in one or both of the jaw members. The knife is selectively advanceable through both the first and second electrically conductive plates of one or both of the jaw members. In yet other aspects, the knife is connected to an energy source and is independently activatable. In aspects, the knife may be configured to utilize ultrasonic energy to divide tissue.

In accordance with another aspect of the present disclosure, a forceps includes a housing having one or more shaft members extending therefrom configured to support an end effector assembly at a distal end thereof. The end effector includes first and second opposing jaw members each having a first electrically conductive plate associated with a first portion of the jaw member and a second electrically conductive plate associated with a second portion of the jaw member. One or more of the first and second jaw members is pivotable relative to the other about a pivot such that the jaw members are selectively movable between an open position wherein the jaw members are spaced relative to one another and a closed position for grasping tissue therebetween. The first and second electrically conductive plates of the first and second jaw members are disposed in opposition relative to one another. A first switch is included that is adapted to connect to a first mode of an electrosurgical generator and is configured to energize the first opposing electrically conductive plates on the first and second jaw members to seal tissue upon activation thereof. A second switch is included that is adapted to connect to a tissue monitoring system and is configured to obtain feedback from the tissue disposed between the second opposing electrically conductive plates on the first and second jaw members.

In aspects according to the present disclosure, an insulator is disposed between the first and second electrically conductive plates of one or both jaw members. In other aspects, a knife assembly is operably associated with the housing and is configured to advance a knife through a knife channel defined in one or both of the jaw members. The knife is selectively advanceable through the first electrically conductive plate of one or both jaw members.

### BRIEF DESCRIPTION OF THE DRAWINGS

Various aspects of the present disclosure are described herein with reference to the drawings wherein like reference numerals identify similar or identical elements:

FIG. 1 is a side, perspective view of an open forceps including opposing shaft members and an end effector assembly disposed at a distal end thereof according to an embodiment of the present disclosure;

FIG. 2 is a side, perspective view of a endoscopic forceps including an end effector assembly disposed at a distal end thereof according to another embodiment of the present disclosure;

FIG. 3 is a side view of the forceps of FIG. 1;

FIG. 4A is an enlarged, distal perspective view of the end effector assembly of FIG. 1 including first and second jaw members;

FIG. 4B is an enlarged, top view of the first jaw member including a seal plate with a seal surface and a surface for dissecting tissue;

FIG. 5A is an enlarged, top view of the first jaw member of another embodiment according to the present disclosure including a seal plate with a seal surface and an advanced energy delivery or monitoring zone;

FIG. 5B is a side view of the end effector assembly of FIG. 5A; and

FIG. 5C is a side, perspective view of another embodiment according to the present disclosure including a seal surface and combination bipolar and nerve monitoring zone.

### DETAILED DESCRIPTION

Throughout the description, like reference numerals and letters indicate corresponding structure throughout the several views. Also, any particular feature(s) of a particular exemplary embodiment may be equally applied to any other exemplary embodiment(s) of this specification as suitable. In other words, features between the various exemplary embodiments described herein are interchangeable as suitable, and not exclusive.

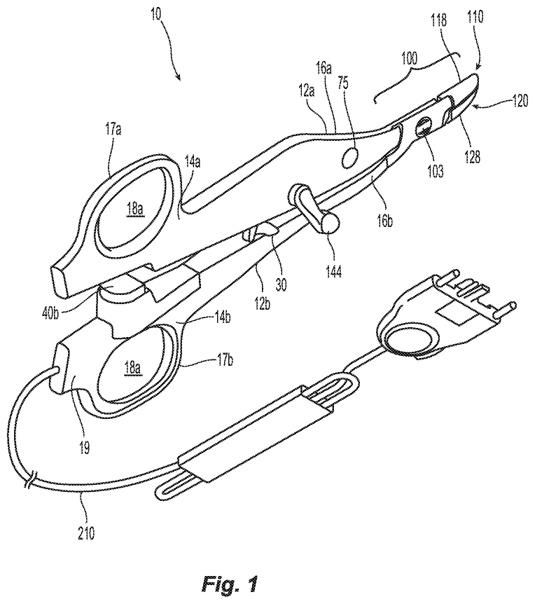

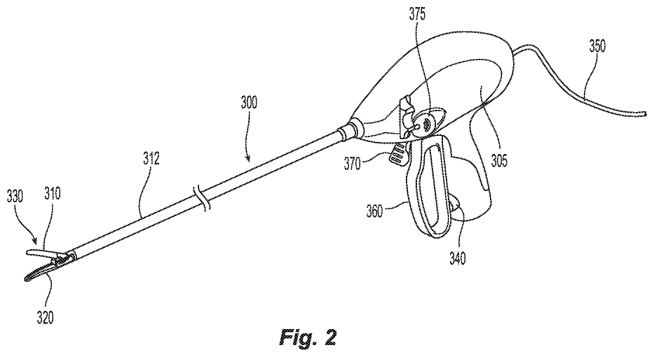

Referring now to FIG. 1, an open forceps 10 contemplated for use in connection with traditional open surgical procedures is shown. For the purposes herein, either an open instrument, e.g., forceps 10, or an endoscopic forceps 300 (FIG. 2) may be utilized in accordance with the present disclosure. Obviously, different electrical and mechanical connections and considerations apply to each particular type of forceps; however, the novel aspects with respect to the end effector assembly and its operating characteristics remain generally consistent with respect to both the open and endoscopic configurations.

With continued reference to FIG. 1, forceps 10 includes two elongated shafts 12a and 12b, each having a proximal end 14a and 14b, and a distal end 16a and 16b, respectively. Forceps 10 further includes an end effector assembly 100 attached to distal ends 16a and 16b of shafts 12a and 12b, respectively. End effector assembly 100 includes a pair of opposing jaw members 110 and 120 that are pivotably connected about a pivot 103. Each shaft 12a and 12b includes a handle 17a and 17b disposed at the proximal end 14a and 14b thereof. Each handle 17a and 17b defines a finger hole 18a and 18b therethrough for receiving a finger of the user. As can be appreciated, finger holes 18a and 18b facilitate movement of the shaft members 12a and 12b relative to one another between a spaced-apart position and an approximated position, which, in turn, pivot jaw members 110 and 120 from an open position, wherein the jaw members 110 and 120 are disposed in spaced-apart relation relative to one another, to a closed position, wherein the jaw members 110 and 120 cooperate to grasp tissue therebetween.

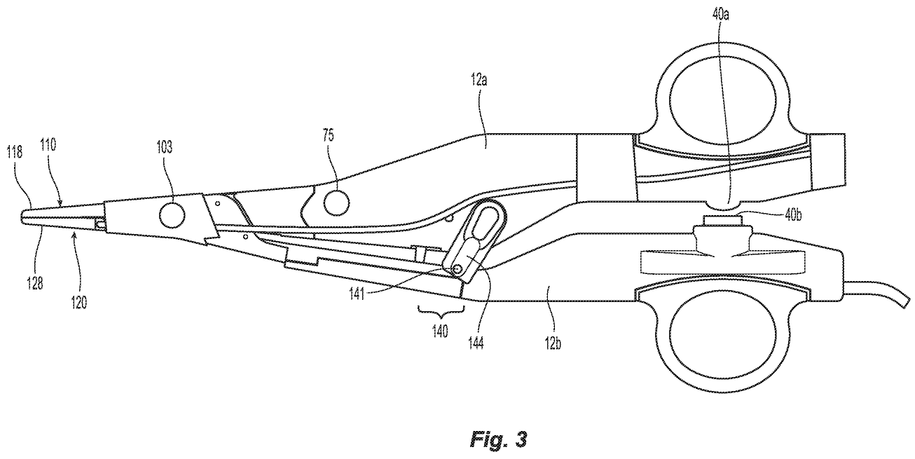

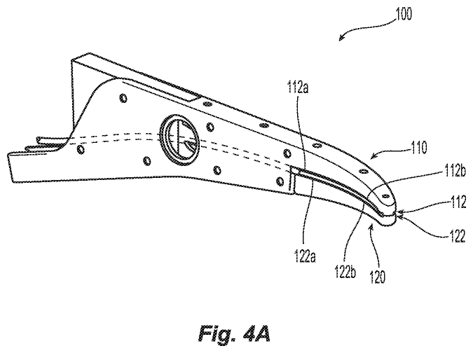

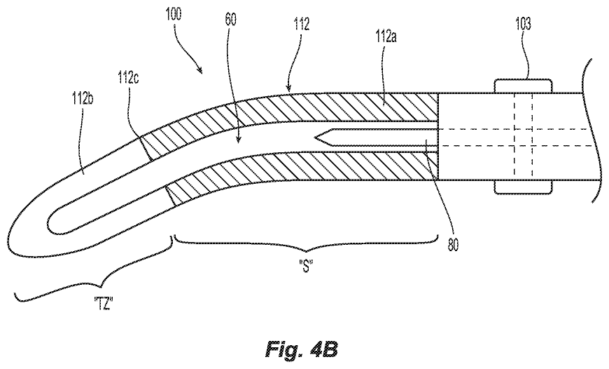

Continuing with reference to FIG. 1, one of the shafts, e.g., shaft 12b, includes a proximal shaft connector 19 that is configured to connect the forceps 10 to a source of electrosurgical energy such as an electrosurgical generator (not shown). Proximal shaft connector 19 secures an electrosurgical cable 210 to forceps 10 such that the user may selectively apply electrosurgical energy to the tissue sealing plates 112 and 122 (see FIGS. 3-4) of jaw members 110 and 120, respectively. More specifically, cable 210 includes one or more wires (not shown) extending therethrough that has sufficient length to extend through one of the shaft members, e.g., shaft member 12b, in order to provide electrical energy to at least a portion of at least one of the sealing plates 112, 122 of jaw members 110, 120, respectively, of end effector assembly 100, e.g., upon activation of activation switch 40b (See FIGS. 1 and 3). Alternatively, forceps 10 may be configured as a battery-powered instrument.

Activation switch 40b is disposed at proximal end 14b of shaft member 12b and extends therefrom towards shaft member 12a. A corresponding surface 40a is defined along shaft member 12a toward proximal end 14a thereof and is configured to actuate activation switch 40b (See FIGS. 1 and 2). More specifically, upon approximation of shaft members 12a, 12b, e.g., when jaw members 110, 120 are moved to the closed position, activation switch 40b is moved into contact with, or in close proximity of, surface 40a. Upon further approximation of shaft members 12a, 12b, e.g., upon application of a pre-determined closure force to jaw members 110, 120, activation switch 40b is advanced further into surface 40a to depress activation switch 40b. Activation switch 40b controls the supply of electrosurgical energy to jaw members 110, 120 such that, upon depression of activation switch 40b, electrosurgical energy is supplied to at least a portion of sealing surface 112 and/or at least a portion of sealing surface 122 of jaw members 110, 120, respectively, to seal or otherwise treat tissue grasped therebetween. Other more standardized activation switches are also contemplated, e.g., finger switch, toggle switch, foot switch, etc.

Referring now to FIG. 2, endoscopic forceps 300 is shown and includes a housing 305 having an elongated shaft 312 extending therefrom that supports an end effector assembly 330 at a distal end thereof. End effector assembly 330 is similar to end effector assembly 100 described above and includes opposing jaw members 310 and 320 that cooperate to grasp and treat tissue. The jaw members 310 and 320 are movable via a handle 360 and a trigger 370 is used to advance the knife, e.g., knife 80. An activation switch 340 is activated when the handle 360 is fully actuated and the jaw members 310 and 320 are closed about tissue. Energy is supplied to the forceps 300 via a power cable 350 that connects to a generator (not shown). A second activation switch 375 is included to activate the advanced functions of the forceps 300. Details relating to the various activation functions, energy modalities and basic operation of the forceps 300 are explained below with reference to forceps 10.

Referring now to FIG. 3, in conjunction with FIG. 1, forceps 10 (or forceps 300) may further include a knife assembly 140 disposed within one of the shaft members, e.g., shaft member 12a, and a knife channel 60 (FIG. 4B) defined within one or both of jaw members 110, 120, respectively, to permit reciprocation of a knife 80 (FIG. 4B) therethrough. Knife assembly 140 includes a rotatable trigger 144 coupled thereto that is rotatable about a pivot 141 for advancing the knife 80 from a retracted position within shaft member 12a, to an extended position wherein the knife 80 extends into knife channel 60 to divide tissue grasped between jaw members 110, 120. In other words, axial rotation of trigger 144 effects longitudinal translation of knife 80. Other trigger assemblies are also contemplated.

Each jaw member 110, 120 of end effector assembly 100 may include a jaw frame having a proximal flange extending proximally therefrom. The jaw frames are engagable with one another to permit pivoting of jaw members 110, 120 about a common pivot 103 relative to one another between the open position and the closed positions upon movement of shaft members 12a, 12b (FIG. 1) relative to one another between the spaced-apart and approximated or closed positions. Proximal flanges of jaw members 110, 120 also connect jaw members 110, 120 to the respective shaft members 12b, 12a thereof, e.g., via welding.

Jaw members 110, 120 may each further include an insulative housing 118 and 128, respectively, that is configured to receive a plate 112, 122, respectively, thereon and that is configured to electrically isolate the plates 112, 122 from the remaining components of the respective jaw members 110, 120 (FIG. 3). As explained in more detail below, portions or sections of the plates 112, 122 may be conductive, non-conductive or configured to monitor tissue.

As shown in FIG. 3, plates 112, 122 of jaw members 110, 120 are disposed in opposed relation relative to one another such that, upon movement of jaw members 110, 120 to the closed position, tissue is grasped between plates 112, 122, respectively, thereof. Accordingly, in use, electrosurgical energy may be supplied to one or both of plates 112, 122 and conducted through tissue to seal tissue grasped therebetween and/or knife 80 may be advanced through knife channels 60 of jaw members 110, 120 to cut tissue grasped therebetween.

FIGS. 4A and 4B show enlarged views of the end effector assembly 100 of forceps 10 including opposing jaw members 110 and 120. Each jaw member, e.g., jaw member 110, includes a plate 112 that extends along the length thereof that defines a knife channel 60 therein. Jaw member 120 includes plate 122. Knife channel 60 is configured to reciprocate knife 80 along the knife channel 60 to cut tissue disposed between the jaw members 110 and 120 upon actuation thereof. Knife channel 60 may be defined in one or both jaw members 110 and 120 depending upon a particular purpose.

Each plate, e.g., plate 112, is divided into proximal and distal sections, 112a and 112b, respectively. Plate section 112a cooperates with corresponding plate section 122a on jaw member 120 and plate section 112b cooperates with corresponding plate section 122b on jaw member 120. As explained in more detail below, plate sections 112b and 122b may be conductive or non-conductive depending upon a particular purpose. Opposing plate sections, e.g., 112a, 122a and 112b, 122b, each cooperate to treat tissue in a different fashion. More particularly, plate sections 112a and 122a cooperate to seal tissue disposed within the proximal section of the jaw members 110 and 120, e.g., the seal zone “S”, upon activation of switch 40b (or switch 340 of FIG. 2). Tissue disposed between plate sections 112b and 122b, e.g., the tip zone “TZ”, will remain untreated upon activation of switch 40b. More particularly, tissue disposed between plate sections 112b and 122b can be finely dissected and cut via “cold dissection” which involves actuating and advancing the knife 80 through untreated or unsealed tissue in the tip zone “TZ”.

An insulator 112c may be disposed between plate sections, e.g., between plate sections 112a and 112b, to electrically or thermally insulate plate section 112b during activation. During use, a surgeon may opt to seal tissue by positioning the tissue between the plate sections 112a and 122a and squeezing the handles 12a and 12b which progressively grasps the tissue and activates the switch 40b in the same range of motion. Switch 40b may be activated in a more conventional manner, e.g., manual activation or via a footswitch. If a surgeon wishes to dissect and cut tissue without employing electrical energy, the surgeon can position the tissue between plate sections 112b and 122b in the tip zone “TZ”. The surgeon can then squeeze the handles 12a and 12b to firmly grasp the tissue and actuate the trigger 144 to advance the knife 80 through the tissue. This is commonly referred to as “cold dissection”.

With respect to the particular embodiment of the forceps 10 shown in FIGS. 1 and 3, activation of the switch 40b by virtue of the handles 12a and 12b closing will not transfer energy to the tip zone “TZ” due to the position of the insulator 112c disposed therebetween. Moreover and in this instance, the knife 80 may be configured to transition through the knife channel 60 without contacting any energized surface in the plate sections 112a and 122a to avoid unintentionally energizing the knife 80. Alternatively, the knife 80 may include insulated portions (not shown) to accomplish this purpose.

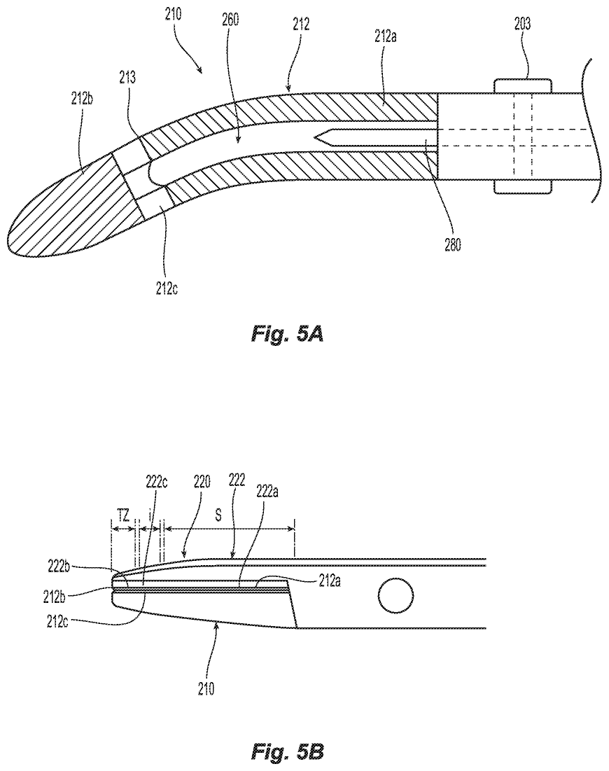

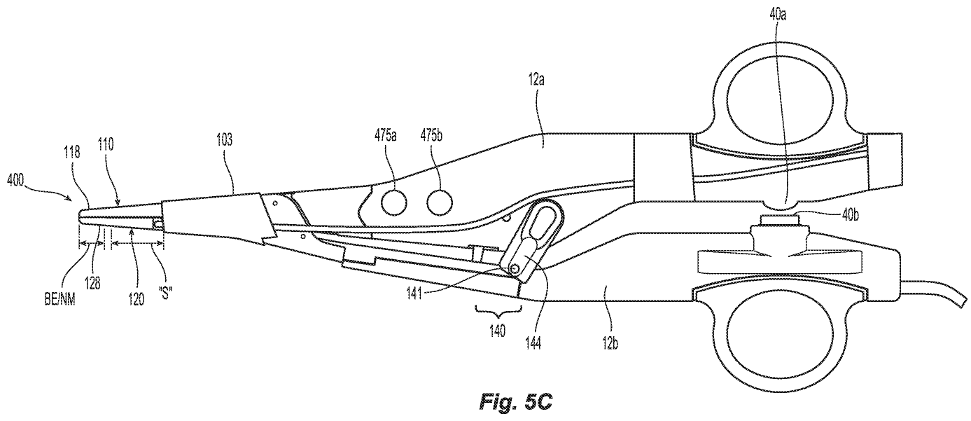

Referring now to FIGS. 5A and 5B, other jaw member and plate section configurations are envisioned. More particularly, jaw member 210 includes similar features to jaw member 110 and cooperates with an opposing jaw member 220 to treat tissue as described below. Jaw member 210 includes a plate 212 having plate sections 212a and 212b disposed thereon separated by an insulator 212c. Plate section 212a is similar to plate section 112a and is configured to cooperate with opposing plate section 222a to seal tissue in the seal zone “S” upon activation of switch 40b. Plate section 212b is configured to cooperate with opposing plate section 222b and treat or monitor tissue within the tip zone “TZ” upon activation of a second switch 75 (See FIGS. 1 and 3).

More particularly, in one embodiment, upon activation of switch 75 the plate sections 212b and 222b of the tip zone “TZ” are configured to conduct bipolar energy through tissue disposed therebetween to coagulate the tissue (versus seal the tissue placed in plate sections 212a and 222a). In this instance, the surgeon can opt for sealing tissue or coagulating tissue depending upon the position of the tissue on the plates 212 and 222. Insulator 212c prevents the different energy modalities and energy algorithms from interfering with one another when switches 40b and 75 are independently activated.

In another embodiment, activation of switch 75 provides a monitoring function, e.g., tissue monitoring, nerve monitoring or tissue identification. In other words, the tip zone “TZ” of the forceps 10 may be utilized for multiple functions during a surgical procedure. Tissue or nerves may be monitored or quickly identified at any time by the surgeon simply by placing the tissue within the tip zone “TZ” and activating the switch 75 to provide feedback to a monitoring system. This allows the surgeon to quickly assess tissue types and avoid critical structures during a procedure without having to swap instruments.

For example, intraoperative nerve monitoring systems may be operably connected to the tip zone “TZ” to enable surgeons to identify, confirm, and monitor motor nerve function to help reduce the risk of nerve damage during various procedures, including ENT and general surgeries. Nerve monitoring systems such as the NIM-Response® 3.0 and NIM-Neuro® 3.0 sold by Medtronic offer advanced nerve monitoring technology with an easy-to-use interface. NIM® systems monitor EMG activity from multiple muscles. If there is a change in nerve function, the NIM system may provide audible and visual warnings to help reduce the risk of nerve damage during various surgical procedures.

Forceps 10 and 300 may be configured to operably connect to one or more generators (not shown) or a generator and a tissue or nerve monitoring system (not shown). A single generator is also contemplated that provides electrosurgical energy in multiple modalities with various algorithms, e.g., vessel sealing and bipolar coagulation, as well as nerve/tissue monitoring. Alternatively, multiple generators may be utilized in conjunction with a nerve or tissue monitoring system, e.g., the NIM-Response® 3.0 or the NIM-Neuro® 3.0 systems.

FIG. 5A also shows a knife stop 213 disposed at a distal-most end of the knife channel 260. Knife stop 213 prevents the knife 280 from advancing into the tip zone “TZ” when actuated and avoids accidental dissection of critical tissue structures before or after monitoring.

As shown in FIG. 5C, a combination forceps 400 is contemplated where one or multiple switches may be utilized; one switch, e.g., switch 475a, for providing bipolar energy to tissue within the tip zone “BE/NM” and the other switch 475b for enabling tissue or nerve monitoring or identification of tissue within the tip zone “BE/NM”. A third switch, e.g., switch 40b, operates in a similar fashion as described above and provides electrosurgical energy the proximal zone “S” according to the algorithm for sealing tissue.

The distal tip of each jaw member, e.g., jaw member 110, 120 and/or 210, 220, may be tapered and curved to permit fine dissection of tissue structures and to facilitate fine tissue plane dissection for the location of nerves and other critical structures. Tapering the distal tips of the jaw members, e.g., jaw members, 110, 120 and/or 210, 220, also provides better visualization during treatment and manipulation of tissue and reduces thermal spread. The distal tips may be fine but also blunt to allow blunt dissection of the tissue in a so-called “poke and spread” manner.

The knife 80, 280 may be connected to an electrosurgical energy source to facilitate dissection of tissue. In embodiments, the knife 80, 280 may use ultrasonic energy to divide tissue either before or after tissue treatment or to assist in dissection.

The various embodiments disclosed herein may also be configured to work with robotic surgical systems and what is commonly referred to as “Telesurgery.” Such systems employ various robotic elements to assist the clinician and allow remote operation (or partial remote operation) of surgical instrumentation. Various robotic arms, gears, cams, pulleys, electric and mechanical motors, etc. may be employed for this purpose and may be designed with a robotic surgical system to assist the clinician during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of clinicians may prep the patient for surgery and configure the robotic surgical system with one or more of the instruments disclosed herein while another clinician (or group of clinicians) remotely controls the instruments via the robotic surgical system. As can be appreciated, a highly skilled clinician may perform multiple operations in multiple locations without leaving his/her remote console which can be both economically advantageous and a benefit to the patient or a series of patients.

For a detailed description of exemplary medical work stations and/or components thereof, reference may be made to U.S. Patent Application Publication No. 2012/0116416, and PCT Application Publication No. WO2016/025132, the entire contents of each of which are incorporated by reference herein.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, the elements and features shown or described in connection with certain embodiments may be combined with the elements and features of certain other embodiments without departing from the scope of the present disclosure, and that such modifications and variations are also included within the scope of the present disclosure. Accordingly, the subject matter of the present disclosure is not limited by what has been particularly shown and described.

What is claimed is:

1. A forceps, comprising:
a housing;
at least one shaft member extending from the housing configured to support an end effector assembly at a distal end thereof, the end effector including first and second opposing jaw members each including a first plate associated with a first portion of the jaw member and a second plate associated with a second portion of the jaw member, at least one of the first and second jaw members pivotable relative to the other about a pivot such that the jaw members are selectively movable between an open position wherein the jaw members are spaced relative to one another and a closed position for grasping tissue therebetween, wherein the first and second plates of the first and second jaw members are disposed in opposition relative to one another, the first plates of the first and second jaw members adapted to connect to an electrosurgical generator such that, upon activation thereof, the first plates energize tissue disposed therebetween; and
a knife assembly operably associated with the housing and configured to advance a knife through a knife channel defined in at least one of the jaw members, the knife selectively advanceable through both the first and second plates of the at least one jaw member.

2. The forceps according to claim 1, wherein the second plate is non-conductive.

3. The forceps according to claim 1, further comprising an insulator disposed between the first and second plates of at least one jaw member.

4. The forceps according to claim 1, wherein the second plate is adapted to connect to a tissue monitoring system.

5. The forceps according to claim 1, further comprising a first switch adapted to connect to the electrosurgical generator and configured to energize the first opposing plates on the first and second jaw members to seal tissue upon activation thereof, and a second switch adapted to connect to a tissue monitoring system and configured to obtain feedback from the tissue disposed between the second opposing plates on the first and second jaw members.

6. The forceps according to claim 5, wherein the tissue monitoring system provides feedback relating to at least one of nerves, critical tissue structures, or tissue type.

7. The forceps according to claim 1, further comprising a first switch adapted to connect to a first mode of the electrosurgical generator and configured to energize the first opposing plates on the first and second jaw members to seal tissue upon activation thereof, and a second switch adapted to connect to a second mode of the electrosurgical generator and configured to coagulate tissue disposed between the second opposing plates on the first and second jaw members upon activation thereof.

8. A forceps, comprising:
a housing;
at least one shaft member extending from the housing configured to support an end effector assembly at a distal end thereof, the end effector including first and second opposing jaw members each including a first electrically conductive plate associated with a first portion of the jaw member and a second electrically conductive plate associated with a second portion of the jaw member, at least one of the first and second jaw members pivotable relative to the other about a pivot such that the jaw members are selectively movable between an open position wherein the jaw members are spaced relative to one another and a closed position for grasping tissue therebetween, wherein the first and second electrically conductive plates of the first and second jaw members are disposed in opposition relative to one another;
a first switch adapted to connect to a first mode of an electrosurgical generator and configured to energize the first opposing electrically conductive plates on the first and second jaw members to seal tissue upon activation thereof;
a second switch adapted to connect to a tissue monitoring system and configured to obtain feedback from the tissue disposed between the second opposing electrically conductive plates on the first and second jaw members; and
a third switch adapted to connect to a second mode of the electrosurgical generator and configured to energize the second opposing electrically conductive plates on the first and second jaw members to coagulate tissue upon activation thereof.

9. The forceps according to claim 8 further comprising a knife assembly operably associated with the housing and configured to selectively advance a knife through a knife channel defined in at least one of the jaw members, the knife selectively advanceable through both the first and second electrically conductive plates of the at least one jaw member.

10. The forceps according to claim 8, further comprising an insulator disposed between the first and second plates of at least one jaw member.

11. The forceps according to claim 9, wherein the knife is connected to an energy source and is independently activatable.

12. The forceps according to claim 9, wherein the knife uses ultrasonic energy to divide tissue.

13. A forceps, comprising:
a housing;
at least one shaft member extending from the housing configured to support an end effector assembly at a distal end thereof, the end effector including first and second opposing jaw members each including a first electrically conductive plate associated with a first portion of the jaw member and a second electrically conductive plate associated with a second portion of the jaw member, at least one of the first and second jaw members pivotable relative to the other about a pivot such that the jaw members are selectively movable between an open position wherein the jaw members are spaced relative to one another and a closed position for grasping tissue therebetween, wherein the first and second electrically conductive plates of the first and second jaw members are disposed in opposition relative to one another;
a first switch adapted to connect to a first mode of an electrosurgical generator and configured to energize the first opposing electrically conductive plates on the first and second jaw members to seal tissue upon activation thereof; and
a second switch adapted to connect to a tissue monitoring system and configured to obtain feedback from the tissue disposed between the second opposing electrically conductive plates on the first and second jaw members.

14. The forceps according to claim 13, further comprising an insulator disposed between the first and second electrically conductive plates of at least one jaw member.

15. The forceps according to claim 13 further comprising a knife assembly operably associated with the housing and configured to advance a knife through a knife channel defined in at least one of the jaw members, the knife selectively advanceable through the first electrically conductive plate of the at least one jaw member.
